(19) United States
(12) Patent Application Publication
(10) Pub. No.: US 2024/0417762 A1
(43) Pub. Date: Dec. 19, 2024

(54) MICROORGANISMS AND METHODS FOR PRODUCING REUTERIN

(71) Applicant: Wisconsin Alumni Research Foundation, Madison, WI (US)

(72) Inventors: Jan Peter van Pijkeren, Madison, WI (US); Zhiying Wang, Madison, WI (US)

(73) Assignee: Wisconsin Alumni Research Foundation, Madison, WI (US)

(21) Appl. No.: 18/743,376

(22) Filed: Jun. 14, 2024

Related U.S. Application Data
(60) Provisional application No. 63/521,210, filed on Jun. 15, 2023.

(51) Int. Cl. C12P 7/52; C12N 9/12

(52) U.S. Cl. CPC C12P 7/52; C12N 9/12; C12Y 207/11001

(57) ABSTRACT

Microorganisms and methods for producing reuterin. The microorganisms express a phage protein from Limosilactobacillus reuteri, Limosilactobacillus mucosae, or Limosilactobacillus oris, or a homolog thereof, that enhances the production of reuterin. The methods can include culturing the microorganisms to thereby produce the reuterin.

### STATEMENT REGARDING FEDERALLY SPONSORED RESEARCH

This invention was made with government support under AT011202 awarded by the National Institutes of Health. The government has certain rights in the invention.

### SEQUENCE LISTING

The instant application contains a Sequence Listing which has been submitted in XML format and is hereby incorporated by reference in its entirety. The XML copy, created on May 28, 2024, is named USPTO--240528--09824525-P230254US02--APP--SEQ_LIST.xml and is 34,709 bytes in size.

### FIELD OF THE INVENTION

The invention is directed to microorganisms and methods for producing reuterin, specifically, by expressing a phage protein in microorganisms.

### BACKGROUND

Reuterin is an important industrial chemical that is produced by a number of microorganisms during fermentation of glycerol. Reuterin has broad-spectrum antimicrobial properties (Vollenweider et al. 2004) and contributes to the probiosis of a number of probiotics such as Limosilactobacillus reuteri by inhibiting gut invaders (Casas et al. 2000). In addition, reuterin is widely used as a food preservative (Schaefer et al. 2010) and an auxiliary therapeutic agent (De Weirdt et al. 2012) and is also an important precursor for several industrial chemicals (Stevens et al. 2011). Previous research has optimized the fermentation conditions of microbes to increase the productivity (Mishra et al. 2018, Begunova et al. 2020). In addition, genes in the propanediol utilization (pdu) operon have been overexpressed to convert glycerol to reuterin more efficiently (Dishisha et al. 2014). However, strategies to further enhance the production of reuterin are needed.

### SUMMARY OF THE INVENTION

One aspect of the invention is directed to recombinant microorganisms.

In some versions, the microorganisms comprise one or more modifications with respect to a corresponding microorganism not comprising the one or more modifications. In some versions, the one or more modifications comprise a recombinant orf14 gene encoding an Orf14 protein comprising an amino acid sequence of Orf14 of Limosilactobacillus reuteri (SEQ ID NO:2), Orf14 of Limosilactobacillus mucosae (SEQ ID NO:4), or Orf14 of Limosilactobacillus oris (SEQ ID NO: 6), or an amino acid sequence of a homolog of any of the foregoing.

In some versions, the Orf14 protein comprises an amino acid sequence having at least 80% sequence identity to SEQ ID NO:2, SEQ ID NO:4, or SEQ ID NO:6.

In some versions, the Orf14 protein comprises an amino acid sequence having at least 95% sequence identity to SEQ ID NO:2, SEQ ID NO:4, or SEQ ID NO:6.

In some versions, the recombinant orf14 gene comprises a promoter and a coding sequence, wherein the promoter is heterologous to the coding sequence.

In some versions, the recombinant microorganism is a bacterium.

In some versions, the recombinant microorganism is commensal to human gastrointestinal tract.

In some versions, the recombinant microorganism comprises a glycerol dehydratase. In some versions, the recombinant microorganism comprises a propanediol utilization gene set.

In some versions, the recombinant microorganism and the corresponding microorganism produce reuterin.

In some versions, the recombinant microorganism exhibits enhanced production of reuterin with respect to the corresponding microorganism.

In some versions, the recombinant microorganism is from a genus selected from the group consisting of Anaerobutyricum, Bacillus, Blautia Citrobacter, Clostridium, Enterobacter, Enterococcus, Flavonifractor, Klebsiella, Lactobacillus, Limosilactobacillus, Listeria, Pediococcus, Salmonella, Streptococcus, Terrisporobacter, and Yersina. In some versions, the recombinant microorganism is L. reuteri.

In some versions, the recombinant microorganism comprises an inactivation of at least one biologically active prophage present in the corresponding microorganism.

In some versions, the recombinant microorganism is L. reuteri and comprises an inactivation of at least one of Φ1 and Φ2.

In some versions, the recombinant microorganism comprises an inactivation of each biologically active prophage present in the corresponding microorganism.

In some versions, the recombinant microorganism is L. reuteri and comprises an inactivation of each of Φ1 and Φ2.

Another aspect of the invention is directed to methods of producing reuterin.

In some versions, the methods comprise culturing a recombinant microorganism of the invention thereby produce reuterin.

In some versions, the culturing is performed in vivo. In some versions, the culturing is performed in a gastrointestinal tract. Some versions further comprise introducing the recombinant microorganism to a (the) gastrointestinal tract. Some versions further comprise orally administering the recombinant microorganism.

In some versions, culturing is performed in vitro.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Schematic representation of two prophage genomes: Limosilactobacillus reuteri 1 (LRΦ1) and Φ2 (LRΦ2) (Oh et al. 2019). Genes are coded based on their predicted functions.

FIG. 2. Predicted protein structure of the Limosilactobacillus reuteri Orf14.

FIG. 3. Schema of prophage deletion, showing location of oligonucleotides on the prophage and bacterial chromosome to screen for integrated or deleted prophages (Oh et al. 2019).

FIG. 4. Schema of the pdu pathway in reuterin-producing microorganisms, showing the synthesis and excretion (production) of reuterin (3-HPA) through the action of glycerol dehydratase (GDH, PduCDE). Other enzymes in the pdu pathway include: propionaldehyde dehydrogenase (PduP, also referred to as CoA-acylating propionaldehyde dehydrogenase), which catalyzes the generation of 3-hydroxypropionyl-CoA (3-HP CoA) from reuterin; phosphate propanoyltransferase (PduL, also known as phosphotransacylase), which catalyzes the generation of 3-hydroxypropionyl phosphate (3-HP-P) from 3-HP CoA; propionate kinase (PduW), which catalyzes the generation of 3-hydroxypropionic acid (3-HP) from 3-HP-P, and 1,3-propanediol oxidoreductase (PduQ, 1,3-PD oxidoreductase), which catalyzes the generation of 1,3-propanediol (1,3-PD) from reuterin. See Chen et al. 2017, Sun et al. 2022, and Liang et al. 2021.

FIGS. 5A-5E. Reuterin production of L. reuteri ATCC PTA-6475 and its variants during the growth. Overnight cells were inoculated to reach an initial OD 0.1 in mMRS supplemented with 100 mM glucose and 40 mM glycerol. Supernatant was collected every hour during the growth at 37° C. and the reuterin concentrations were determined. The number of cells of each strain at each time point was normalized to an OD of 3. Data are shown as means±standard deviations for three independent experiments. Statistical significance was determined by unpaired t-test. * P<0.05; **** P<0.0001. Data are shown as means±standard deviations for three independent experiments. In FIGS. 5C and 5D, “orf14” refers to orf14 from L. reuteri. In FIG. 5E, the origins of the orf14 genes (L. mucosae or L. reuteri) are indicated.

### DETAILED DESCRIPTION OF THE INVENTION

One aspect of the invention is directed to recombinant microorganisms. “Recombinant microorganism” refers to a microorganism that comprises a recombinant nucleic acid, a recombinant gene, or a recombinant polypeptide. A recombinant nucleic acid or polypeptide is one comprising a sequence that is not naturally occurring. A recombinant gene is a gene that comprises a recombinant nucleic acid sequence, is present within a microorganism in which it does not naturally occur, and/or is present at a locus (e.g., genetic locus or on an extrachromosomal plasmid) in which it does not naturally occur.

The recombinant microorganisms of the invention can comprise one or more modifications with respect to a corresponding microorganism not comprising the one or more modifications. “Corresponding microorganism” refers to a microorganism of the same species having the same or substantially same genetic and proteomic composition as a recombinant microorganism of the invention, with the exception of genetic and proteomic differences resulting from the modifications described herein for the recombinant microorganisms of the invention. The corresponding microorganism is typically a microorganism from which the recombinant microorganisms of the invention are derived and in which the modifications are introduced. In some versions, the corresponding microorganism is the native version of the recombinant microorganism of the invention, i.e., the unmodified microorganism as found in nature. The terms “microorganism” and “microbe” are used interchangeably herein.

The recombinant microorganisms of the invention preferably comprise a recombinant orf14 gene encoding an Orf14 protein comprising an amino acid sequence of Orf14 of Limosilactobacillus reuteri (SEQ ID NO:2), Orf14 of Limosilactobacillus mucosae (SEQ ID NO: 4), or Orf14 of Limosilactobacillus oris (SEQ ID NO:6), or an amino acid sequence of a homolog of any of the foregoing. As used herein, the designation “orf14” or “orf14 gene” refers to any gene encoding an Orf14 protein of the invention.

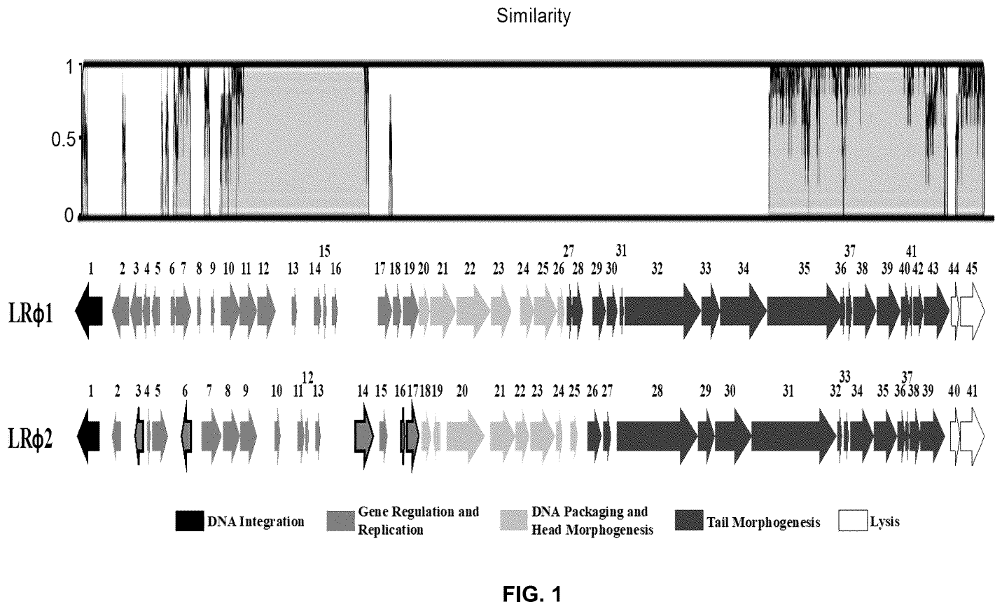

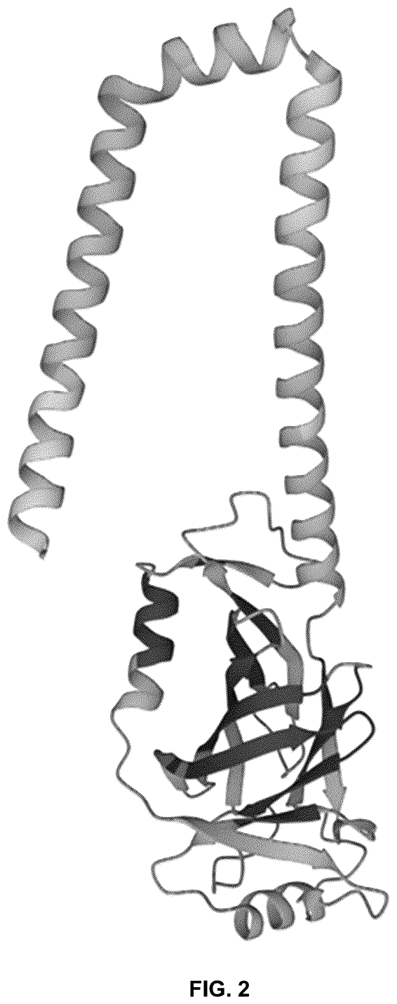

The Limosilactobacillus reuteri Orf14 protein is a protein encoded by 2 of Limosilactobacillus reuteri (Oh et al. 2019) (FIGS. 1 and 2). The amino acid sequence of the Limosilactobacillus reuteri Orf14 protein is SEQ ID NO:2:

(SEQ ID NO: 2)
VIYMNKIKRLCKKYQQVIYTLFAVLVIISVVYFALHGFDSFVKWATTDVG
SVADWAGSIGTILAFVAVIWQQGRQENITRAVHIEESRPRFSLTYTPKPV
LKTRVLFWGNGRTAVNINEILGSRSAENYRFISIENISNNVVYDYSIILK
YHSNDNARVRKDYWSSHGLFPRRSVAIVPKFMGTDKDKIGNYIYDELLVK
FTTPANEVGFFISKNINNAQTDSSFGKSQYYFVRGSHVKRVTAINTDKMI
DVDSSICRKLDKEFDEIIGITSLGEINENGKLS

The coding sequence of the Limosilactobacillus reuteri Orf14 protein is SEQ ID NO:1:

(SEQ ID NO: 1)
GTGATATATATGAATAAAATAAAAAGGTTATGTAAAAAATACCAGCAAGT
AATTTATACACTATTTGCTGTCCTAGTTATTATAAGTGTTGTTTATTTTG
CTTTGCATGGGTTTGATTCATTTGTAAAGTGGGCAACAACTGATGTGGGG
TCTGTCGCTGATTGGGCTGGAAGTATTGGAACAATTCTTGCTTTTGTTGC
TGTTATATGGCAACAAGGGAGACAAGAAAACATTACAAGAGCTGTTCATA
TCGAAGAATCAAGGCCTAGATTTTCGCTTACATATACACCTAAGCCGGTG
TTGAAAACAAGAGTGTTGTTTTGGGGCAATGGTCGAACGGCAGTTAATAT
AAATGAAATTCTTGGAAGTAGGTCAGCTGAGAATTACAGATTTATTAGTA
TCGAAAATATTTCTAATAATGTAGTTTATGATTATTCCATAATATTAAAG
TATCACTCTAATGATAATGCAAGAGTTAGAAAAGATTATTGGTCAAGTCA
TGGATTATTTCCAAGAAGAAGTGTTGCGATTGTTCCTAAGTTTATGGGCA
CTGATAAAGATAAAATAGGAAATTACATATATGATGAATTACTTGTTAAA
TTTACAACTCCGGCTAATGAAGTAGGATTTTTTATATCGAAGAACATAAA
TAACGCTCAAACAGATTCTAGTTTTGGCAAAAGTCAGTATTATTTTGTAC
GAGGAAGCCACGTTAAGCGTGTAACGGCTATTAACACAGATAAAATGATT
GATGTTGATAGTTCCATTTGTAGGAAATTAGATAAAGAGTTTGACGAAAT
AATTGGTATAACAAGTTTAGGAGAAATAAATGAGAATGGAAAACTTTCTT
AA

The Limosilactobacillus mucosae and Limosilactobacillus oris Orf14 proteins are natural homologs of the Limosilactobacillus reuteri Orf14 protein. The amino acid sequence of the Limosilactobacillus mucosae Orf14 protein is SEQ ID NO:4:

(SEQ ID NO: 4)
MKRVKELCKKYQQVIYTLLAVVAIVGVVYFALHGFNSFIKWATTDVGSVA
DWAGSIGTILAFIAVIWQQGRQENITRAVHVEESRPRFSVTYTPKPQAKT
KMLFWGNNRTLSQINTMLNNRNGENYRFINIENISNNVVYDYSIILKYHF
YDNSLVRKDYWSSHGLFPRRSIIIVPKFLGTGKDKIGNYIYDELLIKFTT
PANEIGFFKMTNVNGNRTDSSFGDSQYYFVRGSHIKRVTAINTDEMIKAD
SPLCRKLDREFSETTGGTNFGAIGEDGKAY

The coding sequence of the Limosilactobacillus mucosae Orf14 protein is SEQ ID NO:3:

(SEQ ID NO: 3)
ATGAAAAGGGTAAAAGAATTATGTAAAAAATATCAGCAAGTAATTTACAC
TTTATTAGCTGTTGTCGCTATTGTAGGTGTTGTTTATTTTGCTTTGCATG
GATTTAATTCATTTATAAAATGGGCAACAACTGATGTAGGCTCTGTTGCT
GATTGGGCTGGGAGTATTGGAACTATCCTTGCTTTTATTGCTGTTATTTG
GCAACAAGGAAGACAAGAAAATATCACACGTGCTGTTCATGTTGAAGAAT
CAAGGCCTAGATTTTCTGTCACGTATACTCCCAAACCTCAAGCAAAAACA
AAAATGTTGTTTTGGGGCAACAATCGAACTTTAAGCCAAATAAATACCAT
GTTAAATAATAGAAATGGTGAAAACTATAGATTTATAAATATAGAAAATA
TTTCAAATAATGTTGTCTATGATTATTCAATAATATTAAAATACCATTTT
TATGATAATTCCTTAGTACGAAAGGACTATTGGTCAAGTCATGGATTGTT
CCCGAGAAGAAGTATCATTATTGTTCCAAAATTTTTAGGGACTGGCAAAG
ACAAGATAGGGAATTATATTTATGATGAACTACTTATTAAATTTACAACT
CCAGCTAATGAAATTGGCTTTTTCAAAATGACGAATGTTAATGGTAATCG
AACAGATTCTAGCTTTGGCGATAGTCAATATTATTTTGTCCGTGGAAGCC
ATATTAAACGAGTAACAGCAATTAATACTGATGAAATGATCAAAGCTGAT
AGTCCACTTTGTAGAAAATTAGATAGAGAATTTAGTGAAACAACCGGTGG
AACAAATTTTGGGGCAATAGGAGAAGATGGAAAAGCTTACTAA

The amino acid sequence of the Limosilactobacillus oris Orf14 protein is (SEQ ID NO:6):

(SEQ ID NO: 6)
MLNNIKSLYSKFKQFIQGALCIVVLIFGFILIKHITKEWTPFITWFHTDH
GSVADWAGSIGTIAAFLAVFWQVKKQGSIERAIDVERSRPRFSVLFSLTL
PKGTRVLYWNRTDNDANNIISNPEEYRFITIQNISSNVIYDFDVILRYHT
LDNSRSRNDFWSTTGVFPKKTVTFIPKFKGKNNDQQYVYDELLVKFTTPA
NEVGFFRMINVNDVKGDLGLGSGRYYFVKGTHIKGVKAINKDRMIKINSD
ECQRFDSLFEDFNYSTNTIEV

The coding sequence of the Limosilactobacillus oris Orf14 protein is (SEQ ID NO:5):

(SEQ ID NO: 5)
ATGTTGAATAATATAAAATCTTTATATTCCAAATTTAAGCAATTTATACA
AGGAGCTTTATGCATAGTTGTACTTATCTTTGGTTTTATTTTAATAAAGC
ATATAACAAAAGAATGGACACCATTTATTACATGGTTTCATACTGATCAT
GGAAGTGTTGCAGATTGGGCAGGGAGTATTGGAACTATTGCAGCGTTTTT
AGCTGTTTTTTGGCAAGTGAAAAAACAAGGAAGTATTGAAAGAGCTATTG
ATGTTGAAAGAAGCAGGCCTAGATTTTCAGTCTTATTTAGTTTAACTTTA
CCTAAAGGTACAAGAGTACTTTATTGGAATAGGACGGATAATGATGCAAA
TAATATAATTAGTAATCCTGAAGAATACCGATTTATTACGATTCAAAACA
TATCTTCAAATGTAATATATGATTTTGATGTGATTTTGAGATATCATACA
TTAGACAATTCACGTAGTCGTAATGACTTTTGGAGTACGACTGGTGTTTT
TCCAAAGAAAACTGTAACTTTTATACCAAAATTTAAAGGGAAAAATAATG
ACCAACAGTATGTGTATGATGAATTATTAGTAAAATTCACGACTCCAGCT
AATGAAGTTGGTTTCTTTCGGATGATTAATGTAAATGATGTAAAAGGTGA
TTTAGGCTTGGGAAGTGGAAGGTATTATTTTGTAAAGGGTACGCATATTA
AGGGAGTCAAGGCAATTAATAAGGATAGAATGATTAAGATTAATAGTGAT
GAATGCCAAAGATTTGATTCATTATTTGAAGACTTTAATTACTCAACAAA
TACTATTGAGGTTTAG

Homologs of the L. reuteri, L. mucosae, and L. oris Orf14 proteins can include variants of these proteins comprising an amino acid sequence with at least 80%, 85%, 90%, 95%, 99%, or 100% sequence identity to SEQ ID NO:2, SEQ ID NO:4, or SEQ ID NO:6. Homologs of the L. reuteri, L. mucosae, and L. oris Orf14 proteins can also include any native homologs of L. reuteri, L. mucosae, and L. oris Orf14 proteins found in other organisms, as well as variants of such native homologs comprising an amino acid sequence with at least 80%, 85%, 90%, 95%, 99%, or 100% sequence identity thereto.

In some versions of the invention, the recombinant gene is configured to be overexpressed in the recombinant microorganism. If a corresponding microorganism endogenously comprises a native orf14 gene, the gene may be modified to exchange or optimize promoters, exchange or optimize enhancers, or exchange or optimize any other genetic element to result in increased expression of the gene. Alternatively, one or more additional copies of the orf14 gene or coding sequence thereof may be introduced to the cell for enhanced expression of the gene product. If a corresponding microorganism does not endogenously comprise a orf14 gene, the gene or coding sequence thereof may be recombinantly introduced for heterologous expression of the gene product. The orf14 gene or coding sequence may be incorporated into the genome of the recombinant microorganism or may be contained on an extra-chromosomal plasmid. The orf14 gene or coding sequence may be introduced to the recombinant microorganism individually or may be included on an operon. Techniques for genetic manipulation are described in further detail below. Accordingly, in some versions of the invention the recombinant orf14 gene comprises a promoter and a coding sequence, wherein the promoter is heterologous to the coding sequence.

In preferred versions of the invention, the recombinant orf14 gene confers enhanced synthesis, excretion, or production of reuterin, such that the recombinant microorganism exhibits enhanced synthesis, excretion, or production of reuterin with respect to the corresponding microorganism. “Reuterin” used as herein refers to 3-hydroxypropanal, 3-hydroxypropionaldehyde:

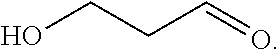

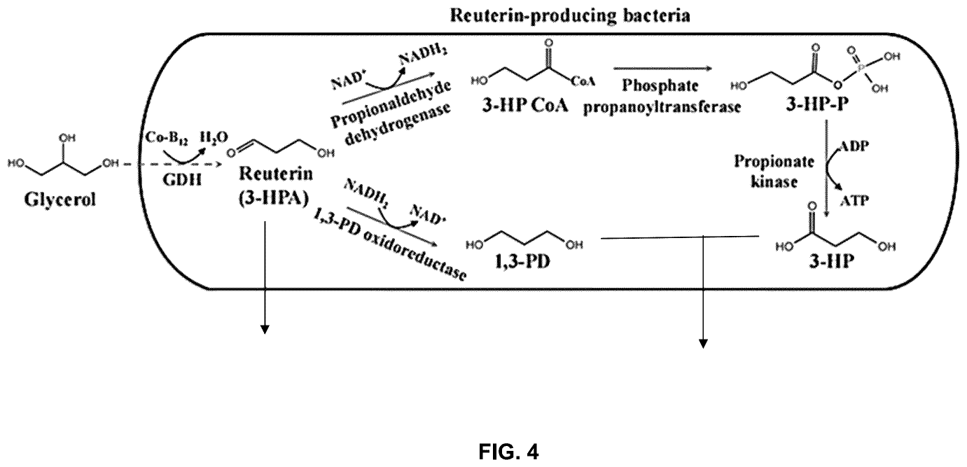

Reuterin is generated in certain microorganisms through the metabolization of glycerol through the action of glycerol dehydratase (GDH, sometimes referred to as PduCDE), typically in the propanediol utilization (pdu) pathway (FIG. 4, FIG. 2 of Chen et al. 2017 and throughout, Sun et al. 2022, and Liang et al. 2021). “Synthesis” (and grammatical variants thereof such as “synthesize”) in this context refers to the generation of reuterin, whether as an intermediate in a metabolic pathway (such as the pdu pathway as described below) or as an end product. “Excretion” (and grammatical variants thereof such as “excrete”) refers to the transport of synthesized reuterin from the interior of a cell to the exterior of the cell. “Production” in this context refers to the synthesis and excretion of reuterin. Methods for determining reuterin production levels are provided in the following examples.

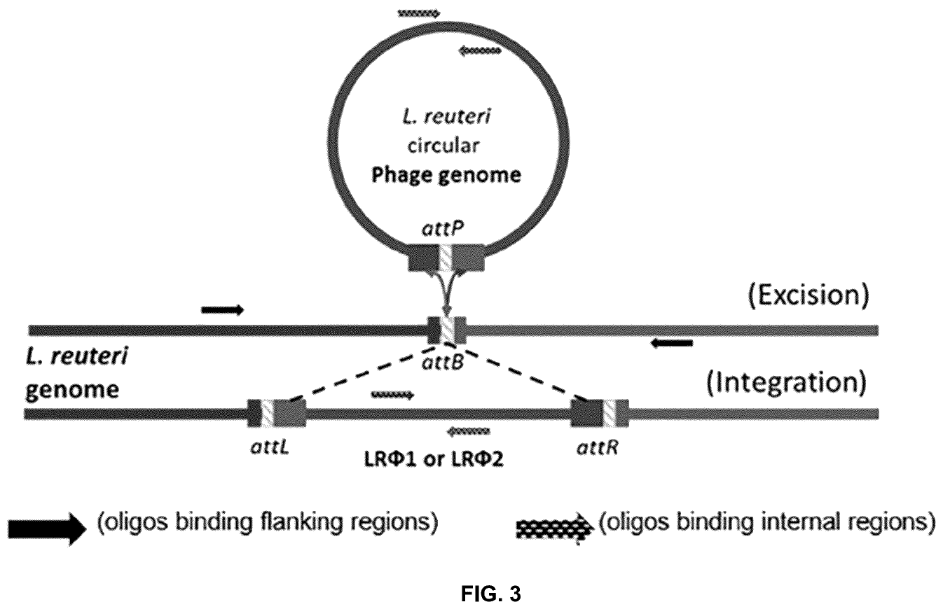

In some versions of the invention, the recombinant microorganism of the invention comprises an inactivation of at least one biologically active prophage present in the corresponding microorganism. In some versions, of the invention, the recombinant microorganism comprises an inactivation of each biologically active lysogenic phage present in the corresponding microorganism. “Prophage” refers to the genetic material of a phage (e.g., bacteriophage) incorporated into the genome of a host organism (such as a recombinant and/or corresponding microorganism of the invention). The term “biologically active” refers to the status of the prophage such that it is able to produce phages if activated. “Inactivation” as used herein with respect to inactivating a biologically active prophage refers to a modification that prevents the ability to produce a phage. The modifications that inactivate a prophage can include genetic mutations to the prophage as it would otherwise exist in the corresponding microorganism or modifications to the non-prophage portions of the genome of the microorganism itself. Such mutations can include substitutions, deletions (whether partial or whole), insertions, etc., of the entire prophage, key genes thereof, or genes or sequences of non-prophage portions of the genome of the microorganism that are responsible for the expression of the prophage genes. The mutations can be introduced by homologous recombination (FIG. 3) or any other method known in the art.

An exemplary microorganism of the invention, L. reuteri, has two biologically active prophages, Φ1 and Φ2 (Oh ct al. 2019) (FIG. 1). In some versions of the invention, the recombinant microorganism is L. reuteri and comprises an inactivation of at least one of Φ1 and Φ2. In some versions of the invention, the recombinant microorganism is L. reuteri and comprises an inactivation of Φ1. In some versions of the invention, the recombinant microorganism is an L. reuteri and comprises an inactivation of Φ2. In some versions of the invention, the recombinant microorganism is an L. reuteri and comprises an inactivation of Φ1 and Φ2. Sec FIG. 3, Oh et al. 2019, and the following examples for exemplary methods of inactivating these prophages.

The recombinant and/or corresponding microorganisms of the invention can comprise bacteria. Bacteria of the invention can include certain commensal or probiotic bacteria, non-commensal bacteria, and other types of bacteria. The bacteria can include non-pathogenic, Gram-positive bacteria capable of anaerobic growth. The bacteria in some cases are viable in the gastrointestinal tract of mammals. The bacteria can be food grade.

Exemplary recombinant and/or corresponding microorganisms of the invention include commensal microorganisms. The commensal microorganisms may be commensal to humans, such as commensal to the human gastrointestinal tract. The commensal microorganisms may comprise commensal bacteria. Exemplary commensal microorganisms to the human gastrointestinal tract include species of the genus Achromobacter, such as Achromobacter spp.; species of the genus Acidaminococcus, such as Acidaminococcus fermentans; species of the genus Acinetobacter, such as Acinetobacter calcoaceticus; species of the genus Actinomyces, such as Actinomyces viscosus and Actinomyces naeslundii; species of the genus Aeromonas; species of the genus Aggregatibacter, such as Aggregatibacter actinomycetemcomitans; species of the genus Anaerobiospirillum; species of the genus, Anaerobutyricum, such as Anaerobutyricum hallii; species of the genus Alcaligenes, such as Alcaligenes faecalis; species of the genus Arachnia, such as Arachnia propionica; species of the genus Bacillus; species of the genus Bacteroides, such as Bacteroides gingivalis, Bacteroides fragilis, Bacteroides intermedius, Bacteroides melaninogenicus, and Bacteroides pneumosintes; species of the genus Bacterionema, such as Bacterionema matruchotii; species of the genus Blautia, such as Blautia obeum; species of the genus Corynebacterium, such as Corynebacterium matruchotii; species of the genus Bifidobacterium; species of the genus Buchnera, such as Buchnera aphidicola; species of the genus Butyriviberio, such as Butyriviberio fibrosolvens; species of the genus Campylobacter, such as Campylobacter coli, Campylobacter sputorum, and Campylobacter upsaliensis; species of the genus Capnocytophaga; species of the genus Clostridium, such as Clostridium difficile and Clostridium sordellii; species of the genus Citrobacter, such as Citrobacter freundii; species of the genus Corynebacterium; species of the genus Cutibacterium, such as Cutibacterium acnes; species of the genus Eikenella, such as Eikenella corrodens; species of the genus Enterobacter, such as Enterobacter cloacae; species of the genus Enterococcus, such as Enterococcus faecalis and Enterococcus faecium; species of the genus Escherichia, such as Escherichia coli; species of the genus Eubacterium; species of the genus Faecalibacterium; species of the genus Flavobacterium; species of the genus Flavonifractor, such as Flavonifractor plautii; species of the genus Fusobacterium, such as Fusobacterium nucleatum; species of the genus Haemophilus, such as Haemophilus parainfluenzae and Haemophilus paraphrophilus; species of the genus Lactobacillus; species of the genus Leptotrichia, such as Leptotrichia buccalis; species of the genus Methanobrevibacter, such as Methanobrevibacter smithii; species of the genus Morganella, such as Morganella morganii; species of the genus Mycobacteria; species of the genus Mycoplasma; species of the genus Micrococcus; species of the genus Mycobacterium, such as Mycobacterium chelonae; species of the genus Neisseria, such as Neisseria sicca; species of the genus Peptococcus; species of the genus Peptostreptococcus; species of the genus Plesiomonas, such as Plesiomonas shigelloides; species of the genus Porphyromonas, such as Porphyromonas gingivalis; species of the genus Propionibacterium; species of the genus Providencia; species of the genus Pseudomonas, such as Pseudomonas aeruginosa; species of the genus Roseburia; species of the genus Rothia, such as Rothia dentocariosa; species of the genus Ruminococcus, such as Ruminococcus bromii; species of the genus Sarcina; species of the genus Staphylococcus, such as Staphylococcus aureus and Staphylococcus epidermidis; species of the genus Streptococcus, such as Streptococcus anginosus, Streptococcus mutans, Streptococcus oralis, Streptococcus pneumoniae, Streptococcus sobrinus, and Streptococcus viridans; species of the genus Torulopsis, such as Torulopsis glabrata; species of the genus Treponema, such as Treponema denticola and Treponema refringens; species of the genus Veillonella; species of the genus Vibrio, such as Vibrio sputorum; species of the genus Wolinella, such as Wolinella succinogenes; and species of the genus Yersinia, such as Yersinia enterocolitica.

Exemplary recombinant and/or corresponding microorganisms of the invention include species of lactic acid bacteria (i.e., species of the order Lactobacillus), such as those from the genera Lactobacillus, Limosilactobacillus, Leuconostoc, Pediococcus, Lactococcus, Streptococcus, Aerococcus, Carnobacterium, Enterococcus, Oenococcus, Fructobacillus, Sporolactobacillus, Tetragenococcus, Vagococcus, and Weissella.

Exemplary recombinant and/or corresponding microorganisms of the invention include species of the Lactobacillus genus. Exemplary species from the Lactobacillus genus include L. acetototerans, L. acidifarinae, L. acidipiscis, L. acidophilus, L. agilis, L. algidus, L. atimentarius, L. amytolyticus, L. amylophilus, L. amylotrophicus, L. amylovorus, L. animatis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. delbrueckii, L. delbrueckii subsp. butgaricus, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hitgardii, L. homohiochii, L. iners, L. ingluviei, L. intestinalis, L. jensenii, L. johnsonii, L. katixensis, L. kefiranofaciens, L. kefiri, L. kimchii, L. kitasatonis, L. kunkeei, L. leichmannii, L. lindneri, L. malefermentans, L. mati, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracollinoides, L. parafarraginis, L. parakefiri, L. paratimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

Exemplary recombinant and/or corresponding microorganisms of the invention include species of the Limosilactobacillus genus. Exemplary species from the Limosilactobacillus genus include L. agrestis, L. albertensis, L. alvi, L. antri, L. balticus, L. caviae, L. coleohominis, L. equigenerosi, L. fastidiosus, L. fermentum, L. frumenti, L. gastricus, L. gorilla, L. ingluviei, L. mucosae, L. oris, L. panis, L. pontis, L. portuensis, L. reuteri, L. rudii, L. secaliphilus, L. urinaemulieris, and L. vaginalis.

Exemplary recombinant and/or corresponding microorganisms of the invention include species of Bifidobacterium. Exemplary species from the Bifidobacterium genus include B. actinocoloniiforme, B. adolescentis, B. aemilianum, B. aerophilum, B. aesculapii, B. amazonense, B. angulatum, B. animalis, B. anseris, B. apousia, B. apri, B. aquikefiri, B. asteroides, B. avesanii, B. biavatii, B. bifidum, B. bohemicum, B. bombi, B. boum, B. breve, B. callimiconis, B. callitrichidarum, B. callitrichos, B. canis, B. castoris, B. catenulatum, B. catulorum, B. cebidarum, B. choerinum, B. choladohabitans, B. choloepi, B. colobi, B. commune, B. criceti, B. crudilactis, B. cuniculi, B. dentium, B. dolichotidis, B. eriksonii, B. erythrocebi, B. eulemuris, B. faecale, B. felsineum, B. gallicum, B. gallinarum, B. globosum, B. goeldii, B. hapali, B. indicum, B. italicum, B. jacchi, B. lemurum, B. leontopitheci, B. longum, B. magnum, B. margollesii, B. merycicum, B. miconis, B. miconisargentati, B. minimum, B. mongoliense, B. moraviense, B. moukalabense, B. myosotis, B. oedipodis, B. olomucense, B. panos, B. parmae, B. platyrrhinorum, B. pluvialisilvae, B. polysaccharolyticum, B. pongonis, B. porcinum, B. primatium, B. pseudocatenulatum, B. pseudolongum, B. psychraerophilum, B. pullorum, B. ramosum, B. reuteri, B. rousetti, B. ruminale, B. ruminantium, B. saguini, B. saguinibicoloris, B. saimiriisciurei, B. samirii, B. santillanense, B. scaligerum, B. scardovii, B. simiarum, B. simiiventris, B. stellenboschense, B. subtile, B. thermacidophilum, B. thermophilum corrig., B. tibiigranuli, B. tissieri corrig., B. tsurumiense, B. urinalis, B. vansinderenii, B. vespertilionis, and B. xylocopae.

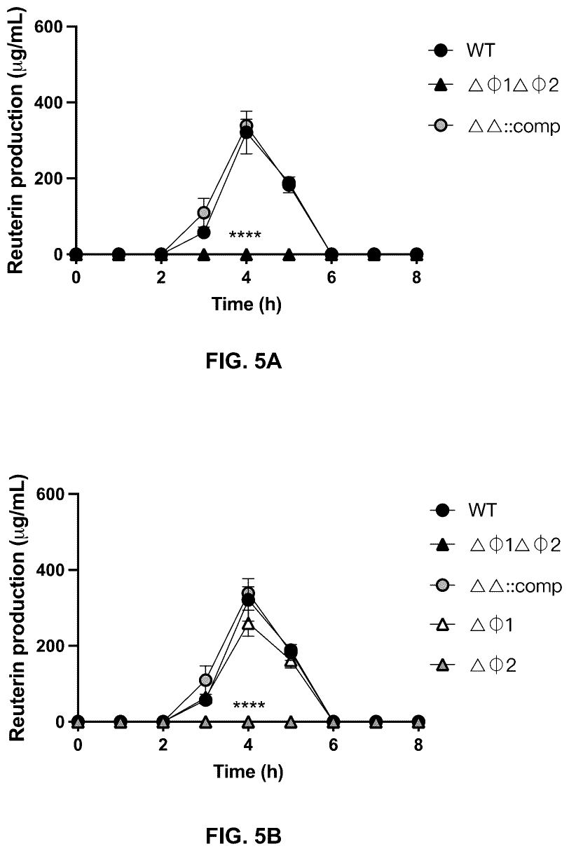

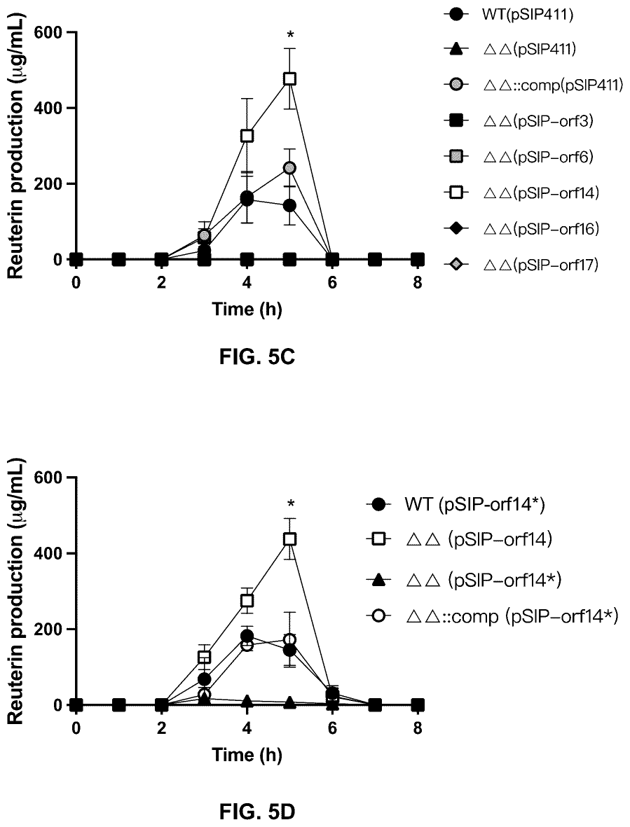

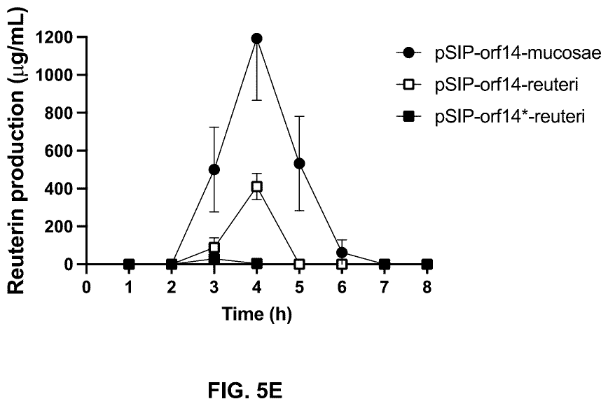

The recombinant and/or corresponding microorganisms of the invention may comprise microorganisms comprising a glycerol dehydratase (GDH). Glycerol dehydratase belongs to the family of lyases, specifically the hydro-lyases, which cleave carbon-oxygen bonds. The systematic name of this enzyme class is glycerol hydro-lyase (3-hydroxypropanol-forming). Other names in common use include glycerol dehydrase, and glycerol hydro-lyase. Glycerol dehydratase hydrolyzes the chemical reaction (EC 4.2.1.30): glycerol⇄3-hydroxypropanal+H₂O. The glycerol dehydratase may be native to the corresponding microorganism or may be engineered into the corresponding and/or recombinant microorganism. The glycerol dehydratase in some microorganisms, including L. reuteri, is PduCDE, which is encoded by pduCDE. The pduCDE genes are often included in the propanediol utilization (pdu) gene cluster (FIGS. 4 and 5), as discussed in further detail below. A number of microorganisms are known to express PduCDE or homologs thereof, including species of Citrobacter, such as Citrobacter freundii; species of Yersinia, such as Yersinia enterocolitica; species of Klebsiella, such as Klebsiella oxytoca and Klebsiella pneumoniae, species of Salmonella, such as Salmonella enterica, species of Terrisporobacter, such as Terrisporobacter glycolicus; species of Clotridium, such as Clostridium perfringens, Clotridium carboxidivorans, and Clotridium pasteuranium; species of Lactobacillus, such as Lactobacillus reuteri, Lactobacillus brevis, and Lactobacillus collinoides; species of Streptococcus, such as Streptococcus sanguinis; species of Enterococcus, such as Enterococcus malodoratus; and species of Listeria, such as Listeria monocytogenes; among others. Scc, e.g., FIG. 2 of Chen et al. 2017, Chen et al. 2017 at large, and O'Brien et al. 2004.

The recombinant and/or corresponding microorganisms of the invention may include microorganisms comprising a propanediol utilization gene set. The propanediol utilization (pdu) gene set is a group of genes present in the propanediol utilization (pdu) gene cluster. The pdu gene cluster is a cluster of genes appearing a large number of microorganisms that carry out a metabolic pathway called the propanediol utilization (pdu) pathway (Chen et al. 2017, Dishisha et al. 2014, Chen et al. 2016) (FIG. 4). The pdu pathway catalyzes dehydration of glycerol to reuterin using glycerol dehydratase (e.g., PduCDE), and further branches to 1,3-propanediol through 1,3-propanediol oxidoreductase (e.g., PduQ), and to 3-hydroxypropionic acid via a series of reactions catalyzed by coenzyme-A acylating propionaldehyde dehydrogenase (e.g., PduP), phosphotransacylasc (e.g., PduL) and propionate kinase (e.g., PduW) (FIG. 4). The pdu gene cluster in some cases can further encode enzymes used to convert 1,2-propanediol to propionic acid and propanol (Bobik et al. 1997). The pdu gene cluster in some cases can further encode structural proteins making up a microcompartment housing the pathway (Sriramulu et al. 2008). For the purposes herein, the phrase “propanediol utilization (pdu) gene set” refers minimally to a set of genes encoding glycerol dehydratase (e.g., PduCDE), 1,3-propanediol oxidoreductase (e.g., PduQ), coenzyme-A acylating propionaldehyde dehydrogenase (e.g., PduP), phosphotransacylasc (e.g., PduL), and propionate kinase (e.g., PduW). In addition to these, the pdu gene set in some embodiments may further include genes encoding any one or more of PduA, PduB, PduF, PduG, PduH, PduK, PduJ, PduM, PduN, PduO, PduS, PduU, PduV, or any other pdu gene shown in FIG. 2 of Chen et al. 2017 or otherwise described therein, Dishisha et al. 2014, or Chen et al. 2016, or any homologs thereof. A number of microorganisms are known to express pdu gene clusters, including species of Citrobacter, such as Citrobacter freundii; species of Yersinia, such as Yersinia enterocolitica; species of Klebsiella, such as Klebsiella oxytoca and Klebsiella pneumoniae, species of Salmonella, such as Salmonella enterica, species of Terrisporobacter, such as Terrisporobacter glycolicus; species of Clotridium, such as Clostridium perfringens, Clotridium carboxidivorans, and Clotridium pasteuranium; species of Lactobacillus, such as Lactobacillus reuteri, Lactobacillus brevis, and Lactobacillus collinoides; species of Streptococcus, such as Streptococcus sanguinis; species of Enterococcus, such as Enterococcus malodoratus; and species of Listeria, such as Listeria monocytogenes; among others. Sec, e.g., FIG. 2 of Chen et al. 2017, Chen et al. 2017 at large, and O'Brien et al. 2004.

In some versions of the invention, recombinant and/or corresponding microorganisms of the invention include microorganisms that synthesize, extracellularly excrete, or produce reuterin. A number of microorganisms are known to produce reuterin natively, including species of Bacillus, Citrobacter, Clostridium, Enterobacter, Enterococcus, Klebsiella, Lactobacillus, Limosilactobacillus, Listeria, Pediococcus, Salmonella, Streptococcus, Terrisporobacter, and Yersina. See Chen et al. 2017, Nakanishi et al. 2002, Sun et al. 2022, Talarico et al. 1988, El-Ziney 1998, and Vollenweider et al. 2004.

In some versions of the invention, recombinant and/or corresponding microorganisms of the invention include microorganisms from a genus selected from the group consisting of Anaerobutyricum, Bacillus, Blautia Citrobacter, Clostridium, Enterobacter, Enterococcus, Flavonifractor, Klebsiella, Lactobacillus, Limosilactobacillus, Listeria, Pediococcus, Salmonella, Streptococcus, Terrisporobacter, and Yersina.

A recombinant and corresponding microorganism used in the following examples is L. reuteri (Limosilactobacillus reuteri formerly referred to as Lactobacillus reuteri). In addition to L. reuteri, other particularly preferred bacteria include L. plantarum (e.g., L. plantarum BAA-793), L. rhamnosus (e.g., L. rhamnosus ATCC 53103)), L. lactis (e.g., L. lactis MG1363), and L. casei. For the purposes herein, the “L.” in the designation “L. reuteri” can refers either to Limosilactobacillus or Lactobacillus.

The invention is also directed to methods of synthesizing, excreting, or producing reuterin. The methods can comprise culturing a recombinant microorganism of the invention to thereby synthesize, excrete, or produce reuterin. As used herein, “culturing” refers to the maintenance of the recombinant microorganisms of the invention in conditions suitable for growth.

In some versions, the culturing is performed in vitro. The in vitro culturing can be performed in liquid culture medium or a solid-phase culture medium (e.g., agar). In some versions, the methods can also comprise isolating the synthesized, excreted, or produced reuterin from the recombinant microorganism. Methods of isolating the synthesized reuterin from the recombinant microorganism can comprise lysing the recombinant microorganism and, optionally, isolating the reuterin from the lysate. Methods of isolating the excreted or produced reuterin from the recombinant microorganism can comprise separating the excreted or produced reuterin from the recombinant microorganism.

In some versions, the culturing is performed in vivo. In some versions, the culturing is performed in a gastrointestinal tract. In some versions, the culturing is performed in a stomach and/or intestines, such as the small intestines. Some versions of the invention can comprise introducing the recombinant microorganism to the gastrointestinal tract. The recombinant microorganism can be introduced to the gastrointestinal tract by any method known in the art. The recombinant microorganism can be administered orally, rectally, or directly into the gastrointestinal tract via a stoma. The recombinant microorganism is preferably administered directly into or upstream of the small intestines, so that the recombinant microorganism ultimately passes through or into the small intestines. The recombinant microorganism can be swallowed or introduced via a tube. The recombinant microorganism can be combined in a composition with a pharmaceutically acceptable excipient, carrier, buffer, stabilizer or other material well known to those skilled in the art. Such materials should be non-toxic and should not interfere with the efficacy of the recombinant microorganism. The precise nature of the carrier or other material may depend on the route of administration. The composition can be liquid, solid, or semi-solid. The composition can comprise a foodstuff or can take the form of a pharmaceutical composition. Those of relevant skill in the art are well able to prepare suitable compositions.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Nucleic acid or gene product (amino acid) sequences of any known gene, including the genes or gene products described herein, can be determined by searching any sequence databases known the art using the gene name or accession number a as search term. Common sequence databases include GenBank (www.ncbi.nlm.nih.gov/genbank/), ExPASy (cxpasy.org), KEGG (www.genomc.jp/kegg/), among others. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 35% 40%, 45% 50%, 55%, 60%, 65%, 70%, 75%, 80%, 85%, 90%, 95%, or 99% or more, can also be used to establish homology. Accordingly, homologs of the genes or gene products described herein include genes or gene products having at least about 30%, 35%, 40%, 45%, 50%, 55%, 60%, 65%, 70%, 75%, 80%, 85%, 90%, 95%, or 99% identity to the genes or gene products described herein. Methods for determining sequence similarity percentages (e.g., BLASTP and BLASTN using default parameters) are described herein and are generally available. The homologous proteins should demonstrate comparable activities and, if an enzyme, participate in the same or analogous pathways. “Orthologs” are genes in different species that evolved from a common ancestral gene by speciation. Normally, orthologs retain the same or similar function in the course of evolution. As used herein “orthologs” are included in the term “homologs”. For sequence comparison and homology determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is a nucleic acid or amino acid sequence corresponding to acsA or other genes or products described herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89: 10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described above (or other algorithms available to persons of skill) or by visual inspection.

The phrase “substantially identical” in the context of two nucleic acids or polypeptides refers to two or more sequences or subsequences that have at least about 60%, about 65%, about 70%, about 75%, about 80%, about 85%, about 90, about 95%, about 98%, or about 99% or more nucleotide or amino acid residue identity, when compared and aligned for maximum correspondence, as measured using a sequence comparison algorithm or by visual inspection. Such “substantially identical” sequences are typically considered to be “homologous”, without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, at least about 250 residues, or over the full length of the two sequences to be compared.

Terms used herein pertaining to genetic manipulation are defined as follows.

Deletion: The removal of one or more nucleotides from a nucleic acid molecule or one or more amino acids from a protein, the regions on either side being joined together.

Derived: When used with reference to a nucleic acid or protein, “derived” means that the nucleic acid or polypeptide is isolated from a described source or is at least 70%, 80%, 90%, 95%, 99%, or more identical to a nucleic acid or polypeptide included in the described source.

Endogenous: An endogenous nucleic acid, gene, gene element (e.g., promoter, enhancer, coding sequence), polypeptide, sequence or any other element in a given cell is one that is naturally occurring in the given cell.

Exogenous: An exogenous nucleic acid, gene, gene element (e.g., promoter, enhancer, coding sequence), polypeptide, sequence or any other element in a given cell is one that is not naturally occurring in the given cell. The term “heterologous” is used herein interchangeably with “exogenous.”

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Gene: “Gene” refers minimally to a coding sequence and a promoter operationally linked to the coding sequence. A gene may additionally include other elements, such as enhancers and silencers.

Introduce: When used with reference to genetic material, such as a nucleic acid, and a cell, “introduce” refers to the delivery of the genetic material to the cell in a manner such that the genetic material is capable of being expressed within the cell. Introduction of genetic material includes both transformation and transfection. Transformation encompasses techniques by which a nucleic acid molecule can be introduced into cells such as prokaryotic cells or non-animal eukaryotic cells. Transfection encompasses techniques by which a nucleic acid molecule can be introduced into cells such as animal cells. These techniques include but are not limited to introduction of a nucleic acid via conjugation, electroporation, lipofection, infection, and particle gun acceleration.

Isolated: An “isolated” biological component (such as a nucleic acid molecule, polypeptide, or cell) has been substantially separated or purified away from other biological components in its original form, such as its native form or the form in which it was originally produced.

Nucleic acid: Encompasses both RNA and DNA molecules including, without limitation, cDNA, genomic DNA, and mRNA. Nucleic acids also include synthetic nucleic acid molecules, such as those that are chemically synthesized or recombinantly produced. The nucleic acid can be double-stranded or single-stranded. Where single-stranded, the nucleic acid molecule can be the sense strand, the antisense strand, or both. In addition, the nucleic acid can be circular or linear.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. An origin of replication is operably linked to a coding sequence if the origin of replication controls the replication or copy number of the nucleic acid in the cell. Operably linked nucleic acids may or may not be contiguous.

Operon: Configurations of separate genes that are transcribed in tandem as a single messenger RNA are denoted as operons. Thus, a set of in-frame genes in close proximity under the transcriptional regulation of a single promoter constitutes an operon. Operons may be synthetically generated using the methods described herein.

Overexpress: When a gene is caused to be transcribed at an elevated rate compared to the endogenous or basal transcription rate for that gene. In some examples, overexpression additionally includes an elevated rate of translation of the gene compared to the endogenous translation rate for that gene. Methods of testing for overexpression are well known in the art, for example transcribed RNA levels can be assessed using rtPCR and protein levels can be assessed using SDS page gel analysis.

Vector or expression vector: An entity comprising a nucleic acid molecule that is capable of introducing the nucleic acid, or being introduced with the nucleic acid, into a cell for expression of the nucleic acid. A vector can include nucleic acid sequences that permit it to replicate in the cell, such as an origin of replication. A vector can also include one or more selectable marker genes and other genetic elements known in the art. Examples of suitable vectors are found below.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below.

Exogenous nucleic acids can be introduced stably or transiently into a cell using techniques well known in the art, including electroporation, calcium phosphate precipitation, DEAE-dextran mediated transfection, liposome-mediated transfection, conjugation, transduction, and the like. For stable transformation, a nucleic acid can further include a selectable marker. Suitable selectable markers include antibiotic resistance genes that confer, for example, resistance to neomycin, tetracycline, chloramphenicol, or kanamycin, genes that complement auxotrophic deficiencies, and the like. (See below for more detail.)

Various embodiments of the invention use an expression vector that includes a heterologous nucleic acid encoding a protein. Suitable expression vectors include, but are not limited to viral vectors, such as baculovirus vectors or those based on vaccinia virus, polio virus, adenovirus, adeno-associated virus, SV40, herpes simplex virus, and the like; phage vectors, such as bacteriophage vectors; plasmids; phagemids; cosmids; fosmids; bacterial artificial chromosomes; Pl-based artificial chromosomes; yeast plasmids; yeast artificial chromosomes; and any other vectors specific for cells of interest.

Useful vectors can include one or more selectable marker genes to provide a phenotypic trait for selection of transformed cells. The selectable marker gene encodes a protein necessary for the survival or growth of transformed cells grown in a selective culture medium. Cells not transformed with the vector containing the selectable marker gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. In alternative embodiments, the selectable marker gene is one that encodes dihydrofolate reductase or confers neomycin resistance (for use in eukaryotic cell culture), or one that confers tetracycline or ampicillin resistance (for use in a prokaryotic cell, such as E. coli).

The coding sequence in the expression vector is operably linked to an appropriate expression control sequence (promoters, enhancers, and the like) to direct synthesis of the encoded gene product. Such promoters can be derived from microbial or viral sources, including CMV and SV40. Depending on the cell/vector system utilized, any of a number of suitable transcription and translation control elements, including constitutive and inducible promoters, transcription enhancer elements, transcription terminators, etc. can be used in the expression vector (see e.g., Bitter et al. (1987) Methods in Enzymology, 153:516-544).

Suitable promoters for use in prokaryotic cells include but are not limited to: promoters capable of recognizing the T4, T3, Sp6, and T7 polymerases; the P_{R} and P_{L} promoters of bacteriophage lambda; the trp, recA, heat shock, and lacZ promoters of E. coli; the alpha-amylase and the sigma-specific promoters of B. subtilis; the promoters of the bacteriophages of Bacillus; Streptomyces promoters; the int promoter of bacteriophage lambda; the bla promoter of the beta-lactamase gene of pBR322; and the CAT promoter of the chloramphenicol acetyl transferase gene. Prokaryotic promoters are reviewed by Glick, J. Ind. Microbiol. 1:277 (1987); Watson et al, Molecular Biology of the Gene, 4th Ed., Benjamin Cummins (1987); and Sambrook et al., In: Molecular Cloning: A Laboratory Manual, 3^{rd} ed., Cold Spring Harbor Laboratory Press (2001).

Non-limiting examples of suitable promoters for use within a eukaryotic cell are typically viral in origin and include the promoter of the mouse metallothionein I gene (Hamer et al. (1982) J. Mol. Appl. Gen. 1:273); the TK promoter of Herpes virus (McKnight (1982) Cell 31:355); the SV40 early promoter (Benoist et al. (1981) Nature (London) 290:304); the Rous sarcoma virus promoter; the cytomegalovirus promoter (Foccking et al. (1980) Gene 45:101); the yeast gal4 gene promoter (Johnston et al. (1982) PNAS (USA) 79:6971; Silver et al. (1984) PNAS (USA) 81:5951); and the IgG promoter (Orlandi et al. (1989) PNAS (USA) 86:3833).

Coding sequences can be operably linked to an inducible promoter. Inducible promoters are those wherein addition of an effector induces expression. Suitable effectors include proteins, metabolites, chemicals, or culture conditions capable of inducing expression. Suitable inducible promoters include but are not limited to the lac promoter (regulated by IPTG or analogs thereof), the lacUV5 promoter (regulated by IPTG or analogs thereof), the tac promoter (regulated by IPTG or analogs thereof), the tre promoter (regulated by IPTG or analogs thereof), the araBAD promoter (regulated by L-arabinose), the phoA promoter (regulated by phosphate starvation), the recA promoter (regulated by nalidixic acid), the proU promoter (regulated by osmolarity changes), the cst-/promoter (regulated by glucose starvation), the tetA promoter (regulated by tetracycline), the cadA promoter (regulated by pH), the nar promoter (regulated by anaerobic conditions), the pr. promoter (regulated by thermal shift), the cspA promoter (regulated by thermal shift), the T7 promoter (regulated by thermal shift), the T7-lac promoter (regulated by IPTG), the T3-lac promoter (regulated by IPTG), the T5-lac promoter (regulated by IPTG), the T4 gene 32 promoter (regulated by T4 infection), the nprM-lac promoter (regulated by IPTG), the VHb promoter (regulated by oxygen), the metallothionein promoter (regulated by heavy metals), the MMTV promoter (regulated by steroids such as dexamethasone) and variants thereof.

Alternatively, a coding sequence can be operably linked to a repressible promoter. Repressible promoters are those wherein addition of an effector represses expression. Examples of repressible promoters include but are not limited to the trp promoter (regulated by tryptophan); tetracycline-repressible promoters, such as those employed in the “TET-OFF”-brand system (Clontech, Mountain View, CA); and variants thereof.

In some versions, the cell is genetically modified with a heterologous nucleic acid encoding a gene product that is operably linked to a constitutive promoter. Suitable constitutive promoters are known in the art and include constitutive adenovirus major late promoter, a constitutive MPSV promoter, and a constitutive CMV promoter.

The relative strengths of the promoters described herein are well-known in the art.

In some versions, the cell is genetically modified with an exogenous nucleic acid encoding a single protein. In other embodiments, a modified cell is one that is genetically modified with exogenous nucleic acids encoding two or more proteins. Where the cell is genetically modified to express two or more proteins, those nucleic acids can each be contained in a single or in separate expression vectors. When the nucleic acids are contained in a single expression vector, the nucleotide sequences may be operably linked to a common control element (e.g., a promoter), that is, the common control element controls expression of all of the coding sequences in the single expression vector.

When the cell is genetically modified with heterologous nucleic acids encoding two or more proteins, one of the nucleic acids can be operably linked to an inducible promoter, and one or more of the nucleic acids can be operably linked to a constitutive promoter. Alternatively, all can be operably linked to inducible promoters or all can be operably linked to constitutive promoters.

Nucleic acids encoding enzymes desired to be expressed in a cell may be codon-optimized for that particular type of cell. Codon optimization can be performed for any nucleic acid by “OPTIMUMGENE”-brand gene design system by GenScript (Piscataway, NJ).

The introduction of a vector into a bacterial cell may be performed by protoplast transformation (Chang and Cohen (1979) Molecular General Genetics, 168:111-115), using competent cells (Young and Spizizen (1961) Journal of Bacteriology, 81:823-829; Dubnau and Davidoff-Abelson (1971) Journal of Molecular Biology, 56:209-221), electroporation (Shigekawa and Dower (1988) Biotechniques, 6:742-751), or conjugation (Kochler and Thorne (1987) Journal of Bacteriology, 169:5771-5278). Commercially available vectors for expressing heterologous proteins in bacterial cells include but are not limited to pZERO, pTrc99A, pUC19, pUC18, pKK223-3, pEX1, pCAL, pET, pSPUTK, pTrxFus, pFastBac, pThioHis, pTrcHis, pTrcHis2, and pLEx, in addition to those described in the following Examples.

Methods for transforming yeast cells with heterologous DNA and producing heterologous polypeptides therefrom are disclosed by Clontech Laboratories, Inc., Palo Alto, Calif., USA (in the product protocol for the “YEASTMAKER”-brand yeast transformation system kit); Reeves et al. (1992) FEMS Microbiology Letters 99:193-198; Manivasakam and Schiestl (1993) Nucleic Acids Research 21 (18): 4414-5; and Ganeva et al. (1994) FEMS Microbiology Letters 121:159-64. Expression and transformation vectors for transformation into many yeast strains are available. For example, expression vectors have been developed for the following yeasts: Candida albicans (Kurtz, et al. (1986) Mol. Cell. Biol. 6:142); Candida maltosa (Kunze et al. (1985) J. Basic Microbiol. 25:141); Hansenula polymorpha (Gleeson et al. (1986) J. Gen. Microbiol. 132:3459) and Roggenkamp et al. (1986) Mol. Gen. Genet. 202:302); Kluyveromyces fragilis (Das et al. (1984) J. Bacteriol. 158:1165); Kluyveromyces lactis (De Louvencourt et al. (1983) J. Bacteriol. 154:737) and Van den Berg et al. (1990) Bio/Technology 8:135); Pichia quillerimondii (Kunze et al. (1985) J. Basic Microbiol. 25:141); Pichia pastoris (Cregg et al. (1985) Mol. Cell. Biol. 5:3376; U.S. Pat. Nos. 4,837,148; 4,929,555); Saccharomyces cerevisiae (Hinnen et al. (1978) Proc. Natl. Acad. Sci. USA 75:1929 and Ito et al. (1983) J. Bacteriol. 153:163); Schizosaccharomyces pombe (Beach et al. (1981) Nature 300:706); and Yarrowia lipolytica (Davidow et al. (1985) Curr. Genet. 10:380-471 and Gaillardin et al. (1985) Curr. Genet. 10:49). Limosilactobacillus reuteri, Limosilactobacillus mucosae, and Limosilactobacillus oris are also known as Lactobacillus reuteri, Lactobacillus mucosae, and Lactobacillus oris, respectively.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### INTRODUCTION

The present examples show the enhancement of reuterin production in active cells by overexpressing the phage protein referred to herein as Orf14 in Limosilactobacillus reuteri as an exemplary microorganism.

### Methods

Characterization and comparison of Φ1 and Φ2 of L. reuteri ATCC PTA-6475

The identification and comparison of prophages Φ1 and Φ2 in L. reuteri ATCC PTA-6475 are described in (Oh et al. 2019). Maps of the phages are shown in FIG. 1.

### Construction of Prophage Deletion Strains (LRΔΦ1, LRΔΦ2, and LRΔΦ1Φ2) and Restoring the Prophages in LRΔΦ1Φ2

L. reuteri strains including a deletion of Φ1 (LRΔΦ1), Φ2 (LRΔΦ2), or both Φ1 and Φ2 (LRΔΦ1Φ2) were generated as described in (Oh et al. 2019). See also FIG. 3.

### Cloning of Individual Φ2 Genes in LRΔ$14$2

Plasmid-based constructs containing genes unique to Φ2 (orf3, orf6, orf14 (SEQ ID NOS: 1-2), orf16, and orf17) were used to assess the impact of these genes on the reuterin production. By PCR (Phusion Hot Start polymerase II, Thermo Scientific) each gene was amplified. See Table 1 for primer pairs. A backbone was amplified with oligos oVPL399 and oVPL400 from pSIP411. Amplicons were fused to backbone by Ligase cycling reaction (de Kok et al. 2014) to generate pSIP-orf3, pSIP-orf6, pSIP-orf14, pSIP-orf16, and pSIP-orf17. After transferring plasmids into Escherichia coli EC1000, the integrity of the constructs was verified by Sanger sequencing. Confirmed plasmid constructs were transformed to LRΔΦ1ΔΦ2 using previously described protocols (Oh et al. 2019) and transformants were confirmed by PCR (Table 1) following by sanger sequencing. The original plasmid pSIP411 was transformed to LR wildtype and LRΔΦ1ΔΦ2, which served as controls.

TABLE 1
Oligonucleotides used in this study.

| Aim | Primer | Sequence (5′→3′) |
|---|---|---|
| pSIP411 backbone | oVPL399 | GGCTAAAATCTCCTTGTAATAGTATTTTATAG |
| | | (SEQ ID NO: 7) |
| | oVPL400 | TAATCTAGACTCGAGGAATTCGGTAC (SEQ ID |
| | | NO: 8) |
| orf3 | oVPL4581 | TTGAACGATGACATTTTACGAGAAAAGA (SEQ |
| | | ID NO: 9) |
| | oVPL4582 | CTAGTCATCGTTCTTTCGATACTTTTTAGG |
| | | (SEQ ID NO: 10) |
| Bridging oligo for | oVPL4583 | ATAAAATACTATTACAAGGAGATTTTAGCCTT |
| orf3 | | GAACGATGACATTTTACGAGAAAAGATA |
| | | (SEQ ID NO: 11) |
| | oVPL4584 | CCTAAAAAGTATCGAAAGAACGATGACTAGT |
| | | AATCTAGACTCGAGGAATTCGGTACCCCG |
| | | (SEQ ID NO: 12) |
| orf6 | oVPL4585 | ATGCAAGATAAAATTGTTGTTAGCTCTTTT |
| | | (SEQ ID NO: 13) |
| | oVPL4586 | CTATTTCCATTTTGACCGCCATTGT (SEQ ID |
| | | NO: 14) |
| Bridging oligo for | oVPL4587 | ATAAAATACTATTACAAGGAGATTTTAGCCAT |
| orf6 | | GCAAGATAAAATTGTTGTTAGCTCTTTT (SEQ |
| | | ID NO: 15) |
| | oVPL4588 | TTCCGACAATGGCGGTCAAAATGGAAATAGT |
| | | AATCTAGACTCGAGGAATTCGGTACCCCG |
| | | (SEQ ID NO: 16) |
| orf14 | oVPL4589 | GTGATATATATGAATAAAATAAAAAGGTTAT |
| | | GTAAAAAATACCA (SEQ ID NO: 17) |
| | oVPL4590 | TTAAGAAAGTTTTCCATTCTCATTTATTTCTCC |
| | | (SEQ ID NO: 18) |
| Bridging oligo for | oVPL4591 | ATAAAATACTATTACAAGGAGATTTTAGCCGT |
| orf14 | | GATATATATGAATAAAATAAAAAGGTTA (SEQ |
| | | ID NO: 19) |
| | oVPL4592 | GAAATAAATGAGAATGGAAAACTTTCTTAAT |
| | | AATCTAGACTCGAGGAATTCGGTACCCCG |
| | | (SEQ ID NO: 20) |
| orf16 | oVPL4593 | ATGGCAGAATTAAGTGAGGCAC (SEQ ID |
| | | NO: 21) |
| | oVPL4594 | TTAATTGTTATTCAACTTATCATCAATCATCTT |
| | | ACG (SEQ ID NO: 22) |
| Bridging oligo for | oVPL4595 | ATAAAATACTATTACAAGGAGATTTTAGCCAT |
| orf16 | | GGCAGAATTAAGTGAGGCACGGCGCAAA |
| | | (SEQ ID NO: 23) |
| | oVPL4596 | ATGATTGATGATAAGTTGAATAACAATTAATA |
| | | ATCTAGACTCGAGGAATTCGGTACCCCG (SEQ |
| | | ID NO: 24) |
| orf17 | oVPL4597 | ATGCCAAGGTATAGAAGATGTAGACA (SEQ ID |
| | | NO: 25) |
| | oVPL4598 | TTATTTTTGGTGCATCAATAGAACGATTTT |
| | | (SEQ ID NO: 26) |
| Bridging oligo for | oVPL4599 | ATAAAATACTATTACAAGGAGATTTTAGCCAT |
| orf17 | | GCCAAGGTATAGAAGATGTAGACAACCT |
| | | (SEQ ID NO: 27) |
| | oVPL4600 | AAAATCGTTCTATTGATGCACCAAAAATAATA |
| | | ATCTAGACTCGAGGAATTCGGTACCCCG (SEQ |
| | | ID NO: 28) |
| Screening | oVPL659 | TGCCCCGTTAGTTGAAGAAG (SEQ ID NO: 29) |
| | oVPL660 | ATTCTGCTCCCGCCCTTATG (SEQ ID NO: 30) |
| pSIP-orf14* | oVPL4690 | TTAAGCAACAAAAGCAAGAATTGTTCCAATA |
| | | CTTC (SEQ ID NO: 31) |
| | oVPL4691 | TGAGTTATATGGCAACAAGGGAGACAAGAAA |
| | | ACAT (SEQ ID NO: 32) |
| MAMA oligo for | oVPL4723 | GTCTCCCTTGTTGCCATATAACTCATTA (SEQ |
| orf14* | | ID NO: 33) |

Construction of pSIP-orf14*

Two in frame stop codons were incorporated into pSIP-orf14 to generate pSIP-orf14*, which loses the function of Orf14 but has the same transcriptional level as pSIP-orf14. By standard blunt-end ligation using T4 DNA ligase (Thermo Scientific), oligonucleotideS oVPL4690 and oVPL4691 were used to generate linear pSIP-orf14* from the backbone of pSIP-orf14. After ligation, DNA was transformed into E. coli EC1000. OligonucleotideS oVPL4723 and oVPL659 were used to confirm the mutation by PCR, followed by Sanger sequencing. Confirmed plasmid pSIP-orf14* was further transformed to LR wildtype, LRΔΦ1ΔΦ2, and LR ΔΦ1ΔΦ2:: comp, respectively.

Construction of pSIP-Orf14-Mucosae and pSIP-Orf14-Oris

To compare the impact of orf14 homologues on the reuterin production, the sequences of two orf14 homologues originally from L. mucosae (SEQ ID NOS: 3-4) and L. oris (SEQ ID NOS: 5-6) were codon optimized for expression in L. reuteri using the OPTIMIZER web server (Puigbo et al. 2007) (Table 2), and double-strand DNA was synthesized by GeneWiz. Synthesized DNA fragments were cloned to pSIP411 backbone as described above.

### Determination of Reuterin Formation

To characterize the reuterin production during growth, strains were cultured at 37° C. anacrobically in deMan-Rogosa-Sharpe medium (MRS; 288110, BD BioSciences) for 18 h, and then bacteria were sub-cultured to reach an initial OD₆₀₀ of 0.1 in modified MRS (10 g peptone, 10 g beef extract, 5 g yeast extract, 2 g ammonium citrate dibasic, 2 g K2HPO4, 0.1 g MgSO4, 0.05 g MnSO4, and 1.0 g Tween 80 per liter) supplemented with 100 mM glucose. Cultures were incubated anaerobically at 37° C. Strains harboring pSIP411 or its variants were grown in mMRS supplemented with 5 μg/mL erythromycin. Where applicable, induction peptide (10 ng/ml) (MAGNSSNFIHKIKQIFTHR (SEQ ID NO:34)) was added. Samples were collected every hour up to 8 hours. Cell density was monitored using GENESYS 20 visible spectrophotometer (Thermo Scientific) and reuterin production was measured as described in Zhang et al. 2020. Briefly, cell-free supernatants (50 μL) were mixed with 37.5 μL tryptophan (Sigma-Aldrich) in a 96 well plate, followed by an addition of 150 μL 37% HCl (Thermo scientific). After incubating at 37° C. for 20 min, OD₅₆₀ was measured by GloMax plate reader (Promega). Two-fold dilutions of acrolein (06-722-501, ThermoFisher) were used to generate a standard curve to quantify reuterin (Doleyres et al. 2005). Reuterin concentration was normalized to OD₆₀₀ of 3 in FIGS. 5A-5D.

### Statistical Analyses

Data were obtained in 3 biological replicates and are expressed as means±standard deviations. Data were analyzed by one-way analysis of variance (ANOVA) using SPSS software, version 21.0 (SPSS Inc., Chicago, IL, USA). The least significant difference (LSD) was used to test the difference among means using a P value of 0.05.

### Results

To understand the role of prophages in reuterin production, we used the previously developed isogenic mutant L. reuteriΔΦ1ΔΦ2 in which both prophage genomes were deleted. We determined that L. reuteriΔΦ14Φ2 does not produce reuterin, which could be restored by complementation of two prophages (FIG. 5A). To determine which prophage drives reuterin production, we tested the single prophage deletion strains, and found that deletion of Φ2 but not deletion of Φ1 abolished reuterin production (FIG. 5B). We therefore hypothesized that Φ2-encoding regulatory genes contribute to reuterin production.

By comparing the two prophage genomes, we identified five unique genes on Φ2 (orf3, orf6, orf14, orf16, and orf17) (FIG. 1), which were cloned into the inducible vector pSIP411. Plasmid pSIP411 and its variants were transferred to L. reuteriΔΦ1ΔΦ2. Wildtype and complementary strains with pSIP411 served as controls. Overexpressing Orf14 in L. reuteriΔΦ1ΔΦ2 not only rescued reuterin production in the L. reuteriΔΦ1ΔΦ2 background, but also resulted in a 3-fold increase (P<0.05) in reuterin production compared to that in the wildtype strain following 5 h incubation (FIG. 5C). The control plasmid in which the coding sequence of orf14 was interrupted by two in-frame stop codons did not restore reuterin production (FIG. 5D).

Two Orf14 homologues were identified in Limosilactobacillus mucosae (SEQ ID NOS: 3 and 4) and Limosilactobacillus oris (SEQ ID NOS: 5 and 6), with amino acid identity of 77.34% and 47.74% respectively by Protein BLAST search in NCBI (National Center for Biotechnology Information). Overexpression of Orf14-mucosae yielded 2.9-fold higher reuterin production in L. reuteri compared to the overexpression of native Orf14 (FIG. 5E), indicating the application of Orf14 in related species (homology>77%).

Taken together, we engineered a hyper reuterin producer L. reuteriΔΦ14Φ2 (pSIP-orf14) by deleting prophages and overexpressing the phage-encoding gene orf14.

### CONCLUSIONS

The high-yield production of reuterin in the engineered strain L. reuteri ΔΦ1ΔΦ2 (pSIP-orf14) highlighted its usefulness as a robust strain for industrial applications. This L. reuteri mutant produces 3-fold more reuterin compared to the control strain. This strain is advantageous because not only is the reuterin production more robust, but reuterin can be harvested from actively growing cells without “leakage” of bacteriophages. The culture media can be directly used for reuterin extraction. This contrasts conventional methods, wherein resting cells need to be harvested, washed, and resuspended in glycerol solution for bioconversion, thereby shortening the process from a two-stage process to a one-stage process. The increased reuterin production by L. reuteri ΔΦ1ΔΦ2 (pSIP-orf14) is also reflected in the antimicrobial activity against ESKAPE pathogens, which may strengthen the probiosis of L. reuteri. Overexpression of orf14 in phage-deleted microbes may also promote the probiotic gut fitness through upregulating the pdu cluster to take advantage of 1,2-propanedial produced by other microbes. The engineering strategies provided herein can be used in biochemistry industry to maximize reuterin production. The resulting strains can be used as advanced probiotic with promoted function and gut fitness.

### REFERENCES

Begunova A V, Rozhkova I V, Shirshova T I, Glazunova O A, Fedorova T V. 2020. Optimization of cultivation conditions for the Lactobacillus reuteri LR1 strain to improve the biosynthesis of bacteriocin-like substances. Appl Biochem Microbiol 56:920-929.
Bobik T A, Havemann G D, Busch R J, Williams D S, Aldrich H C. The propanediol utilization (pdu) operon of Salmonella enterica serovar Typhimurium LT2 includes genes necessary for formation of polyhedral organelles involved in coenzyme B (12)-dependent 1, 2-propanediol degradation. J Bacteriol. 1999; 181:5967-5975.
Bobik T A, Xu Y, Jeter R M, Otto K E, Roth J R. Propanediol utilization genes (pdu) of Salmonella typhimurium: three genes for the propanediol dehydratase. J Bacteriol. 1997; 179:6633-6639.
Casas I A, Dobrogosz W J. 2000. Validation of the probiotic concept: Lactobacillus reuteri confers broad-spectrum protection against disease in humans and animals. Microb Ecol Health Dis 12:247-285.
Chen L, Bromberger P D, Nieuwenhuiys G, Hatti-Kaul R. Redox Balance in Lactobacillus reuteri DSM20016: Roles of iron-dependent alcohol dehydrogenases in glucose/glycerol metabolism. PLOS One. 2016; 11: c0168107.
Chen L, Hatti-Kaul R. Exploring Lactobacillus reuteri DSM20016 as a biocatalyst for transformation of longer chain 1,2-diols: Limits with microcompartment. PLOS One. 2017 Sep. 28; 12 (9): e0185734.
De Weirdt R, Crabbe A, Roos S, Vollenweider S, Lacroix C, van Pijkeren J P, Britton R A, Sarker S, Van de Wiele T, Nickerson C A. 2012. Glycerol supplementation enhances L. reuteri's protective effect against S. Typhimurium colonization in a 3-D model of colonic epithelium. PLOS One 7: e37116.
Dishisha T, Pereyra L P, Pyo S H, Britton R A, Hatti-Kaul R. Flux analysis of the Lactobacillus reuteri propanediol-utilization pathway for production of 3-hydroxypropionaldehyde, 3-hydroxypropionic acid and 1,3-propanediol from glycerol. Microb Cell Fact. 2014; 13:76.
Doleyres Y, Beck P, Vollenweider S, Lacroix C. Production of 3-hydroxypropionaldehyde using a two-step process with Lactobacillus reuteri. Appl Microbiol Biotechnol. 2005 September; 68 (4): 467-74.
de Kok S, Stanton L H, Slaby T, Durot M, Holmes V F, Patel K G, Platt D, Shapland E B, Serber Z, Dean J, Newman J D, Chandran S S. Rapid and reliable DNA assembly via ligase cycling reaction. ACS Synth Biol. 2014 Feb. 21; 3 (2): 97-106.
El-Ziney, M. G., Debevere, J. M. 1998. The effect of reuterin on Listeria monocytogenes and Escherichia coli 0157: H7 in milk and cottage cheese. Journal of Food Protection, 61 (10), 1275-1280.
Liang N, Neužil-Bunešová V, Tejnecký V, Gänzle M, Schwab C. 3-Hydroxypropionic acid contributes to the antibacterial activity of glycerol metabolism by the food microbe Limosilactobacillus reuteri. Food Microbiol. 2021 September; 98: 103720.
Mishra S K, Malik R K, Chawla R, Mishra K K. 2018. Response surface optimization of the cultivation conditions and medium components for maximal reuterin production by L. reuteri BPL-36. J Pharmacogn Phytochem 7:194-200.

Nakanishi K, Tokuda H, Ando T, Yajima M, Nakajima T, Tanaka O, Ohmomo S. Screening of Lactic Acid Bacteria Having the Ability to Produce Reuterin. Japanese Journal of Lactic Acid Bacteria. 2002 37-45.
O'Brien J R, Raynaud C, Croux C, Girbal L, Soucaille P, Lanzilotta W N. Insight into the mechanism of the B12-independent glycerol dehydratase from Clostridium butyricum: preliminary biochemical and structural characterization. Biochemistry. 2004 Apr. 27; 43 (16): 4635-45.
Oh J H, Alexander L M, Pan M, Schueler K L, Keller M P, Attie A D, Walter J, van Pijkeren J P. Dietary Fructose and Microbiota-Derived Short-Chain Fatty Acids Promote Bacteriophage Production in the Gut Symbiont Lactobacillus reuteri. Cell Host Microbe. 2019 Feb. 13; 25 (2): 273-284.e6.
Oh J H, Lin X B, Zhang S, Tollenaar S L, Özçam M, Dunphy C, Walter J, van Pijkeren J P. Prophages in Lactobacillus reuteri Are Associated with Fitness Trade-Offs but Can Increase Competitiveness in the Gut Ecosystem. Appl Environ Microbiol. 2019 Dec. 13; 86 (1): e01922-19.
Pasteris S E, Strasser de Saad A M. Sugar-glycerol cofermentations by Lactobacillus hilgardii isolated from wine. J Agric Food Chem. 2009 May 13; 57 (9): 3853-8.
Puigbo P, Guzman E, Romeu A, Garcia-Vallve S. 2007. OPTIMIZER: a web server for optimizing the codon usage of DNA sequences. Nucleic Acids Res 35: W126-W131.
Ramirez Garcia A, Hurley K, Marastoni G, Diard M, Hofer S, Greppi A, Hardt W D, Lacroix C, Sturla S J, Schwab C. Pathogenic and Commensal Gut Bacteria Harboring Glycerol/Diol Dehydratase Metabolize Glycerol and Produce DNA-Reactive Acrolein. Chem Res Toxicol. 2022 Oct. 17; 35 (10): 1840-1850.
Schaefer L, Auchtung T A, Hermans K E, Whitehead D, Borhan B, Britton R A. 2010. The antimicrobial compound reuterin (3-hydroxypropionaldehyde) induces oxidative stress via interaction with thiol groups. Microbiology 156:1589.
Sriramulu D D, Liang M, Hernandez-Romero D, Raux-Deery E, Lunsdorf H, Parsons J B, et al. Lactobacillus reuteri DSM 20016 produces cobalamin-dependent diol dehydratase in metabolosomes and metabolizes 1,2-propanediol by disproportionation. J Bacteriol. 2008; 190:4559-4567.
Stevens M J A, Vollenweider S, Meile L, Lacroix C. 2011. 1,3-Propanediol dehydrogenases in Lactobacillus reuteri: impact on central metabolism and 3-hydroxypropionaldehyde production. Microb Cell Fact 10:1-9.
Sun M C, Hu Z Y, Li D D, Chen Y X, Xi J H, Zhao C H. Application of the Reuterin System as Food Preservative or Health-Promoting Agent: A Critical Review. Foods. 2022 Dec. 10; 11 (24): 4000.
Suppuram P, Ramakrishnan G G, Subramanian R. An integrated process for the production of 1,3-propanediol, lactate and 3-hydroxypropionic acid by an engineered Lactobacillus reuteri. Biosci Biotechnol Biochem. 2019 April; 83 (4): 755-762.
Talarico, T. L., Casas, I. A., Chung, T. C., Dobrogosz, W. J. 1988. Production and isolation of reuterin, a growth inhibitor produced by Lactobacillus reuteri. Antimicrobial agents and chemotherapy, 32 (12), 1854-1858.
Vollenweider S, Lacroix C. 3-hydroxypropionaldehyde: applications and perspectives of biotechnological production. Appl Microbiol Biotechnol. 2004 March; 64 (1): 16-27.
Zeng A-P, Biebl H, Schlieker H, Deckwer W-D. Pathway analysis of glycerol fermentation by Klebsiella pneumoniae: Regulation of reducing equivalent balance and product formation. Enzyme Microb Technol. 1993; 15:770-779.
Zhang S, Oh J H, Alexander L M, Özçam M, van Pijkeren J P. d-Alanyl-d-Alanine Ligase as a Broad-Host-Range Counterselection Marker in Vancomycin-Resistant Lactic Acid Bacteria. J Bacteriol. 2018 Jun. 11; 200 (13): e00607-17.
Zhang Z, Wang K, Oh J-H, Zhang S, van Pijkeren J-P, Cheng C C, Ren D, Wei H, Gänzle M G, Walter J. 2020. A phylogenetic view on the role of glycerol for growth enhancement and reuterin formation in Limosilactobacillus reuteri. Front Microbiol 11: 601422.

1. A recombinant microorganism comprising one or more modifications with respect to a corresponding microorganism not comprising the one or more modifications, wherein the one or more modifications comprise a recombinant orf14 gene encoding an Orf14 protein comprising an amino acid sequence of Orf14 of Limosilactobacillus reuteri (SEQ ID NO:2), Orf14 of Limosilactobacillus mucosae (SEQ ID NO:4), or Orf14 of Limosilactobacillus oris (SEQ ID NO: 6), or an amino acid sequence of a homolog of any of the foregoing.

2. The recombinant microorganism of claim 1, wherein the Orf14 protein comprises an amino acid sequence having at least 80% sequence identity to SEQ ID NO:2, SEQ ID NO: 4, or SEQ ID NO:6.

3. The recombinant microorganism of claim 1, wherein the Orf14 protein comprises an amino acid sequence having at least 95% sequence identity to SEQ ID NO:2, SEQ ID NO:4, or SEQ ID NO:6.

4. The recombinant microorganism of claim 1, wherein the recombinant orf14 gene comprises a promoter and a coding sequence, wherein the promoter is heterologous to the coding sequence.

5. The recombinant microorganism of claim 1, wherein the recombinant microorganism is a bacterium.

6. The recombinant microorganism of claim 1, wherein the recombinant microorganism is commensal to human gastrointestinal tract.

7. The recombinant microorganism of claim 1, wherein the recombinant microorganism comprises a glycerol dehydratase.

8. The recombinant microorganism of claim 1, wherein the recombinant microorganism comprises a propanediol utilization gene set.

9. The recombinant microorganism of claim 1, wherein the recombinant microorganism and the corresponding microorganism produce reuterin.

10. The recombinant microorganism of claim 1, wherein the recombinant microorganism exhibits enhanced production of reuterin with respect to the corresponding microorganism.

11. The recombinant microorganism of claim 1, wherein the recombinant microorganism is from a genus selected from the group consisting of Anaerobutyricum, Bacillus, Blautia Citrobacter, Clostridium, Enterobacter, Enterococcus, Flavonifractor, Klebsiella, Lactobacillus, Limosilactobacillus, Listeria, Pediococcus, Salmonella, Streptococcus, Terrisporobacter, and Yersina.

12. The recombinant microorganism of claim 1, wherein the recombinant microorganism is L. reuteri.

13. The recombinant microorganism of claim 1, wherein the recombinant microorganism comprises an inactivation of at least one biologically active prophage present in the corresponding microorganism.

14. The recombinant microorganism of claim 13, wherein the recombinant microorganism is L. reuteri and comprises an inactivation of at least one of Φ1 and Φ2.

15. The recombinant microorganism of claim 1, wherein the recombinant microorganism comprises an inactivation of each biologically active prophage present in the corresponding microorganism.

16. The recombinant microorganism of claim 15, wherein the recombinant microorganism is L. reuteri and comprises an inactivation of each of Φ1 and Φ2.

17. A method of producing reuterin, the method comprising culturing the recombinant microorganism of claim 1 to thereby produce reuterin.

18-20. (canceled)

21. The method of claim 17, further comprising orally administering the recombinant microorganism to introduce the recombinant microorganism to a gastrointestinal tract, wherein the culturing is performed in the gastrointestinal tract.

22. The method of claim 17, wherein the culturing is performed in vitro.

23. The recombinant microorganism of claim 1, wherein:
the Orf14 protein comprises an amino acid sequence having at least 95% sequence identity to SEQ ID NO:2, SEQ ID NO:4, or SEQ ID NO:6;
the recombinant orf14 gene comprises a promoter and a coding sequence, wherein the promoter is heterologous to the coding sequence;
the recombinant microorganism is L. reuteri and comprises an inactivation of each of Φ1 and Φ2; and
the recombinant microorganism exhibits enhanced production of reuterin with respect to the corresponding microorganism.


SEQUENCE LISTING (34 sequences)

SEQ ID NO: 1 - DNA, 852 bp, Lactobacillus reuteri
  source 1..852: mol_type = genomic DNA
gtgatatatatgaataaaataaaaaggttatgtaaaaaataccagcaagtaatttatacactatttgctgtcctagttattataagtgttgtttattttgctttgcatgggtttgattcatttgtaaagtgggcaacaactgatgtggggtctgtcgctgattgggctggaagtattggaacaattcttgcttttgttgctgttatatggcaacaagggagacaagaaaacattacaagagctgttcatatcgaagaatcaaggcctagattttcgcttacatatacacctaagccggtgttgaaaacaagagtgttgttttggggcaatggtcgaacggcagttaatataaatgaaattcttggaagtaggtcagctgagaattacagatttattagtatcgaaaatatttctaataatgtagtttatgattattccataatattaaagtatcactctaatgataatgcaagagttagaaaagattattggtcaagtcatggattatttccaagaagaagtgttgcgattgttcctaagtttatgggcactgataaagataaaataggaaattacatatatgatgaattacttgttaaatttacaactccggctaatgaagtaggattttttatatcgaagaacataaataacgctcaaacagattctagttttggcaaaagtcagtattattttgtacgaggaagccacgttaagcgtgtaacggctattaacacagataaaatgattgatgttgatagttccatttgtaggaaattagataaagagtttgacgaaataattggtataacaagtttaggagaaataaatgagaatggaaaactttcttaa

SEQ ID NO: 2 - protein, 283 aa, Lactobacillus reuteri
VIYMNKIKRLCKKYQQVIYTLFAVLVIISVVYFALHGFDSFVKWATTDVGSVADWAGSIGTILAFVAVIWQQGRQENITRAVHIEESRPRFSLTYTPKPVLKTRVLFWGNGRTAVNINEILGSRSAENYRFISIENISNNVVYDYSIILKYHSNDNARVRKDYWSSHGLFPRRSVAIVPKFMGTDKDKIGNYIYDELLVKFTTPANEVGFFISKNINNAQTDSSFGKSQYYFVRGSHVKRVTAINTDKMIDVDSSICRKLDKEFDEIIGITSLGEINENGKLS

SEQ ID NO: 3 - DNA, 843 bp, Lactobacillus mucosae
  source 1..843: mol_type = genomic DNA
atgaaaagggtaaaagaattatgtaaaaaatatcagcaagtaatttacactttattagctgttgtcgctattgtaggtgttgtttattttgctttgcatggatttaattcatttataaaatgggcaacaactgatgtaggctctgttgctgattgggctgggagtattggaactatccttgcttttattgctgttatttggcaacaaggaagacaagaaaatatcacacgtgctgttcatgttgaagaatcaaggcctagattttctgtcacgtatactcccaaacctcaagcaaaaacaaaaatgttgttttggggcaacaatcgaactttaagccaaataaataccatgttaaataatagaaatggtgaaaactatagatttataaatatagaaaatatttcaaataatgttgtctatgattattcaataatattaaaataccatttttatgataattccttagtacgaaaggactattggtcaagtcatggattgttcccgagaagaagtatcattattgttccaaaatttttagggactggcaaagacaagatagggaattatatttatgatgaactacttattaaatttacaactccagctaatgaaattggctttttcaaaatgacgaatgttaatggtaatcgaacagattctagctttggcgatagtcaatattattttgtccgtggaagccatattaaacgagtaacagcaattaatactgatgaaatgatcaaagctgatagtccactttgtagaaaattagatagagaatttagtgaaacaaccggtggaacaaattttggggcaataggagaagatggaaaagcttactaa

SEQ ID NO: 4 - protein, 280 aa, Lactobacillus mucosae
MKRVKELCKKYQQVIYTLLAVVAIVGVVYFALHGFNSFIKWATTDVGSVADWAGSIGTILAFIAVIWQQGRQENITRAVHVEESRPRFSVTYTPKPQAKTKMLFWGNNRTLSQINTMLNNRNGENYRFINIENISNNVVYDYSIILKYHFYDNSLVRKDYWSSHGLFPRRSIIIVPKFLGTGKDKIGNYIYDELLIKFTTPANEIGFFKMTNVNGNRTDSSFGDSQYYFVRGSHIKRVTAINTDEMIKADSPLCRKLDREFSETTGGTNFGAIGEDGKAY

SEQ ID NO: 5 - DNA, 816 bp, Lactobacillus oris
  source 1..816: mol_type = genomic DNA
atgttgaataatataaaatctttatattccaaatttaagcaatttatacaaggagctttatgcatagttgtacttatctttggttttattttaataaagcatataacaaaagaatggacaccatttattacatggtttcatactgatcatggaagtgttgcagattgggcagggagtattggaactattgcagcgtttttagctgttttttggcaagtgaaaaaacaaggaagtattgaaagagctattgatgttgaaagaagcaggcctagattttcagtcttatttagtttaactttacctaaaggtacaagagtactttattggaataggacggataatgatgcaaataatataattagtaatcctgaagaataccgatttattacgattcaaaacatatcttcaaatgtaatatatgattttgatgtgattttgagatatcatacattagacaattcacgtagtcgtaatgacttttggagtacgactggtgtttttccaaagaaaactgtaacttttataccaaaatttaaagggaaaaataatgaccaacagtatgtgtatgatgaattattagtaaaattcacgactccagctaatgaagttggtttctttcggatgattaatgtaaatgatgtaaaaggtgatttaggcttgggaagtggaaggtattattttgtaaagggtacgcatattaagggagtcaaggcaattaataaggatagaatgattaagattaatagtgatgaatgccaaagatttgattcattatttgaagactttaattactcaacaaatactattgaggtttag

SEQ ID NO: 6 - protein, 271 aa, Lactobacillus oris
MLNNIKSLYSKFKQFIQGALCIVVLIFGFILIKHITKEWTPFITWFHTDHGSVADWAGSIGTIAAFLAVFWQVKKQGSIERAIDVERSRPRFSVLFSLTLPKGTRVLYWNRTDNDANNIISNPEEYRFITIQNISSNVIYDFDVILRYHTLDNSRSRNDFWSTTGVFPKKTVTFIPKFKGKNNDQQYVYDELLVKFTTPANEVGFFRMINVNDVKGDLGLGSGRYYFVKGTHIKGVKAINKDRMIKINSDECQRFDSLFEDFNYSTNTIEV

SEQ ID NO: 7 - DNA, 32 bp, synthetic construct
  source 1..32: mol_type = other DNA
ggctaaaatctccttgtaatagtattttatag

SEQ ID NO: 8 - DNA, 26 bp, synthetic construct
  source 1..26: mol_type = other DNA
taatctagactcgaggaattcggtac

SEQ ID NO: 9 - DNA, 28 bp, synthetic construct
  source 1..28: mol_type = other DNA
ttgaacgatgacattttacgagaaaaga

SEQ ID NO: 10 - DNA, 30 bp, synthetic construct
  source 1..30: mol_type = other DNA
ctagtcatcgttctttcgatactttttagg

SEQ ID NO: 11 - DNA, 60 bp, synthetic construct
  source 1..60: mol_type = other DNA
ataaaatactattacaaggagattttagccttgaacgatgacattttacgagaaaagata

SEQ ID NO: 12 - DNA, 60 bp, synthetic construct
  source 1..60: mol_type = other DNA
cctaaaaagtatcgaaagaacgatgactagtaatctagactcgaggaattcggtaccccg

SEQ ID NO: 13 - DNA, 30 bp, synthetic construct
  source 1..30: mol_type = other DNA
atgcaagataaaattgttgttagctctttt

SEQ ID NO: 14 - DNA, 25 bp, synthetic construct
  source 1..25: mol_type = other DNA
ctatttccattttgaccgccattgt

SEQ ID NO: 15 - DNA, 60 bp, synthetic construct
  source 1..60: mol_type = other DNA
ataaaatactattacaaggagattttagccatgcaagataaaattgttgttagctctttt

SEQ ID NO: 16 - DNA, 60 bp, synthetic construct
  source 1..60: mol_type = other DNA
ttccgacaatggcggtcaaaatggaaatagtaatctagactcgaggaattcggtaccccg

SEQ ID NO: 17 - DNA, 44 bp, synthetic construct
  source 1..44: mol_type = other DNA
gtgatatatatgaataaaataaaaaggttatgtaaaaaatacca

SEQ ID NO: 18 - DNA, 33 bp, synthetic construct
  source 1..33: mol_type = other DNA
ttaagaaagttttccattctcatttatttctcc

SEQ ID NO: 19 - DNA, 60 bp, synthetic construct
  source 1..60: mol_type = other DNA
ataaaatactattacaaggagattttagccgtgatatatatgaataaaataaaaaggtta

SEQ ID NO: 20 - DNA, 60 bp, synthetic construct
  source 1..60: mol_type = other DNA
gaaataaatgagaatggaaaactttcttaataatctagactcgaggaattcggtaccccg

SEQ ID NO: 21 - DNA, 22 bp, synthetic construct
  source 1..22: mol_type = other DNA
atggcagaattaagtgaggcac

SEQ ID NO: 22 - DNA, 36 bp, synthetic construct
  source 1..36: mol_type = other DNA
ttaattgttattcaacttatcatcaatcatcttacg

SEQ ID NO: 23 - DNA, 60 bp, synthetic construct
  source 1..60: mol_type = other DNA
ataaaatactattacaaggagattttagccatggcagaattaagtgaggcacggcgcaaa

SEQ ID NO: 24 - DNA, 60 bp, synthetic construct
  source 1..60: mol_type = other DNA
atgattgatgataagttgaataacaattaataatctagactcgaggaattcggtaccccg

SEQ ID NO: 25 - DNA, 26 bp, synthetic construct
  source 1..26: mol_type = other DNA
atgccaaggtatagaagatgtagaca

SEQ ID NO: 26 - DNA, 30 bp, synthetic construct
  source 1..30: mol_type = other DNA
ttatttttggtgcatcaatagaacgatttt

SEQ ID NO: 27 - DNA, 60 bp, synthetic construct
  source 1..60: mol_type = other DNA
ataaaatactattacaaggagattttagccatgccaaggtatagaagatgtagacaacct

SEQ ID NO: 28 - DNA, 60 bp, synthetic construct
  source 1..60: mol_type = other DNA
aaaatcgttctattgatgcaccaaaaataataatctagactcgaggaattcggtaccccg

SEQ ID NO: 29 - DNA, 20 bp, synthetic construct
  source 1..20: mol_type = other DNA
tgccccgttagttgaagaag

SEQ ID NO: 30 - DNA, 20 bp, synthetic construct
  source 1..20: mol_type = other DNA
attctgctcccgcccttatg

SEQ ID NO: 31 - DNA, 35 bp, synthetic construct
  source 1..35: mol_type = other DNA
ttaagcaacaaaagcaagaattgttccaatacttc

SEQ ID NO: 32 - DNA, 35 bp, synthetic construct
  source 1..35: mol_type = other DNA
tgagttatatggcaacaagggagacaagaaaacat

SEQ ID NO: 33 - DNA, 28 bp, synthetic construct
  source 1..28: mol_type = other DNA
gtctcccttgttgccatataactcatta

SEQ ID NO: 34 - protein, 19 aa, synthetic construct
MAGNSSNFIHKIKQIFTHR